(19) United States
(12) Patent Application Publication
(10) Pub. No.: US 2003/0086817 A1
(43) Pub. Date: May 8, 2003

(54) Blood purification system

(76) Inventor: Isaac B. Horton III, Raleigh, NC (US)

Correspondence Address: JINAN GLASGOW, P O BOX 28539, RALEIGH, NC, 276118539

(21) Appl. No.: 10/008,224

(22) Filed: Nov. 6, 2001

(51) Int. Cl. A61L 2/10; A61L 9/20

(52) U.S. Cl. 422/24; 210/764; 210/748

(57) ABSTRACT

An ultraviolet disinfection (UV) system for blood including a UV light-ready blood purifier having at least one portal for receiving UV light input from a UV light source that is removably connected to the blood purifier via a connector at the portal. The portal is positioned to provide a focused, controllable UV light output that has at least one UV dose zone for providing effective sterilization of microorganisms and disinfection within the interior of the blood purifier. Also, an ultraviolet disinfection (UV) system for blood purifiers, the system comprising a light source positioned within a housing that is external a blood purifier and capable of being connected thereto via an optical connector. This system includes at least one source optical component positioned between the light source and the UV light output from the housing, thereby producing a focused, controllable UV light output for providing effective sterilization of microorganisms within the at least one blood purifier. A method for UV disinfection of the interior of blood purifiers is also included in the present invention.

### BACKGROUND OF THE INVENTION

(1) Field of the Invention

The present invention relates generally to a system and method for ultraviolet disinfection and, more particularly, to a system and method for ultraviolet disinfection of blood.

(2) Description of the Prior Art

It is known in the art to use ultraviolet light (UV) for the disinfection treatment of blood. Ultraviolet light, at the germicidal wavelength of 253.7 nanometers, alters the genetic (DNA) material in cells so that bacteria, viruses, molds, algae, and other microorganisms can no longer reproduce. The microorganisms are considered dead, and the risk of disease from them is eliminated. As the air flows past the UV lamps in UV disinfection systems, the microorganisms are exposed to a lethal dose of UV energy. UV dose is measured as the product of UV light intensity times the exposure time within the UV lamp array. Microbiologists have determined the effective dose of UV energy to be approximately about 34,000 microwatt-seconds/cm2 needed to destroy pathogens as well as indicator organisms found in wastewater. Typical prior art disinfection systems and devices emit UV light at approximately 254 nm, which penetrates the outer cell membrane of microorganisms, passes through the cell body, reaches the DNA and alters the genetic material of the microorganism, destroying it without chemicals by rendering it unable to reproduce.

Ultraviolet light is classified into three wavelength ranges: UV-C, from about 200 nanometers (nm) to about 280 nm; UV-B, from about 280 nm to about 315 nm; and UV-A, from about 315 nm to about 400 nm. Generally, UV light, and in particular, UV-C light is “germicidal,” i.e., it deactivates the DNA of bacteria, viruses and other pathogens and thus destroys their ability to multiply and cause disease, effectively resulting in sterilization of the microorganisms. Specifically, UV “C” light causes damage to the nucleic acid of microorganisms by forming covalent bonds between certain adjacent bases in the DNA. The formation of these bonds prevents the DNA from being read correctly, and the organism is neither able to produce molecules essential for life process, nor is it able to reproduce. In fact, when an organism is unable to produce these essential molecules or is unable to replicate, it dies. UV light with a wavelength of approximately between about 250 to about 260 nm provides the highest germicidal effectiveness. While susceptibility to UV light varies, exposure to UV energy for about 20 to about 34 milliwatt-seconds/cm² is adequate to deactivate approximately 99 percent of the pathogens.

Bacterial contamination of blood is a deadly problem that can frequently result in the death of the recipient. 182 deaths from blood transfusions were reported to the U.S. Food and Drug Administration from 1986 to 1991. 16 percent of these deaths were linked to bacterial contamination. There are lab tests to screen donated blood for HIV, hepatitis and other viruses, but none that look for bacteria. Therefore, it is unknown how many of the 20 million pints of blood and blood products used in transfusions each year are contaminated with bacteria. Blood can become contaminated even if the donor is not septicemic. For example, the needle used to siphon blood from a donor can pick up bacteria from the skin. A core of skin is caught inside the needle as the needle is pushed through the skin. The pressure of the blood then pushes the core into the bag.

The most common bacteria found so far are ones, which can grow in cold temperatures, and thus can grow in blood and blood products stored in refrigerators. A more serious problem is contamination of platelets. Once separated from the blood, platelets must be stored at room temperature, which is a good environment for bacteria to grow.

Thus, a blood sterilization process is needed that can sterilize blood in a rapid, effective, and inexpensive manner.

### SUMMARY OF THE INVENTION

The present invention is directed to a UV purification system and method for treating blood.

One object of the present invention is to provide a UV disinfection system for treating blood configured and arranged to function effectively with at least one UV light source or lamp.

Another object of the present invention is to provide a UV-ready blood purifier that is designed to accept a UV light source input for the purpose of sterilization of microorganisms.

Another object of the present invention includes presentation of the UV light source detached from and remotely connectable with the blood purifier via fiber optic, UV transmission lines.

Accordingly, one aspect of the present invention is to provide a UV disinfection system for treating blood configured and arranged to function effectively with at least one UV light source or lamp.

Another aspect of the present invention is to provide a UV-ready blood purifier that is designed to accept a UV light source input for the purpose of sterilization of microorganisms.

Another aspect of the present invention is to provide presentation of the UV light source detached from and remotely connectable with the blood purifier via fiber optic, UV transmission lines, and including the use of optical components.

These and other aspects of the present invention will become apparent to those skilled in the art after a reading of the following description of the preferred embodiment according to the present invention when considered with the drawings.

### BRIEF DESCRIPTION OF THE DRAWINGS

FIG. 1 is a schematic diagram of the complete UV blood disinfection system.

FIG. 2 is a representation of a vertical riser configuration (VRC).

### DETAILED DESCRIPTION OF THE PREFERRED EMBODIMENTS

In the following description, like reference characters designate like or corresponding parts throughout the several views. Also in the following description, it is to be understood that such terms as “forward,” “rearward,” “front,” “back,” “right,” “left,” “upwardly,” “downwardly,” and the like are words of convenience and are not to be construed as limiting terms.

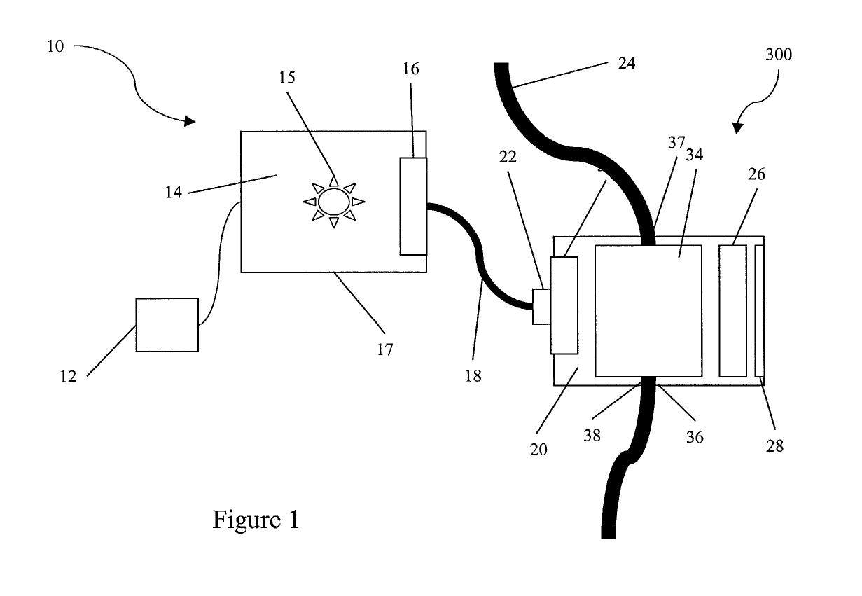

Referring now to the drawings in general, the illustrations are for the purpose of describing a preferred embodiment of the invention and are not intended to limit the invention thereto. FIG. 1 shows a schematic diagram of a UV blood disinfection system, generally described as 10. In the preferred embodiment, a power supply 12 powers a UV light source 14. The UV light source is composed of a UV lamp 15, source optical components 16, and a housing 17. UV light generated by the UV lamp contained within the housing is focused and controlled by the means of the source optical components into at least one UV transmission line 18 that connects to the blood purifier 20 at a portal 22, which may alternatively be at least one portal if more than one light input is desired, thus transmitting UV light to the blood. The blood purifier portal is equipped with optical components, or portal optics, 32 that further control the UV light at the blood purifier 20 in order to provide additional focus and/or control of the UV light for the disinfection of the blood 24. The blood purifier is composed of a dose zone 34 and a housing 36. The dose zone can include a dose delivery device. The dose zone and the housing may be equipped with UV reflective optical components, or interior optics 26, and may also be composed of a UV reflective interior surface and/or coating 28. For longevity as well as UV reflectivity, the interior surfaces may be made of a UV reflective material selected from the group consisting of UV reflective metals, e.g., stainless steel, aluminum, or the like. In the preferred embodiment, the blood purifier is made to be disposable for single-use applications. Additionally, the contribution of the reflectance of internal surfaces to the efficacy of the system can be capitalized upon by incorporating UV reflective materials and reflection enhancing two- and three-dimensional design into the blood purifier. Moreover, additional surfaces to enhance reflectance may be added to the purifier zone. More particularly, the blood purifier and other components form an integrated 2- and 3-dimensional design that incorporates UV-reflectant materials, design, and surfaces that advantageously enhance the efficacy of the system.

While generally regarding the UV light source and configuration according to the present invention, the preferred embodiment includes a UV light source that is remotely connectable to the blood purifier via at least one fiber optic transmission line. Additionally, the preferred embodiment of the present invention includes at least one optical component positioned between the UV light source and the UV light source system output point. Advantageously, the use of optical components enables the system to maximize the intensity, focus, and control of the UV light rays at the output for any given UV light source or lamp. Also, optical components, including but not limited to reflectors, shutters, lenses, splitters, mirrors, rigid and flexible light guides, homogenizer or mixing rods, manifolds and other couplers, filters, color wheels, and the like, can be utilized in combination to achieve the desired control and output. Additionally, optical component such as gratings, dichroic filters, focalizers, gradient lenses, gradient reflectors, off-axis lenses, and off-axis reflectors may be used. All UV transmissive optical components included in the present invention are made of UV-transmissive material and all UV-reflective optical components included in the present invention are made of UV-reflective material. The fiber optic lines may include quartz fibers, side-emitting fibers, glass fibers, acrylic fibers, liquid core fibers, hollow-core fibers, core sheath fibers, dielectric coaxial fibers, or a combination of fibers.

With regard to lenses, several embodiments are considered to be within the scope of the present invention. Imaging lenses, such as a parabolic lens, and non-imaging lenses, such as gradient lenses, may be used to focus and control light output. More particularly, a gradient lens collects light through a collecting opening and focuses it to an area smaller than the area of the collecting opening. This concentration is accomplished by changing the index of refraction of the lens along the axis of light transmission in a continuous or semi-continuous fashion, such that the light is “funneled”to the focus area by refraction. An example of gradient lens technology is the Gradium® Lens manufactured by Solaria Corporation. Alternatively, a toroidal reflector, as described in U.S. Pat. No. 5,836,667, is used. In this embodiment, a UV radiation source, such as an arc lamp, is located at a point displaced from the optical axis of a concave toroidal reflecting surface. The concave primary reflector focuses the radiation from the source at an off-axis image point that is displaced from the optical axis. The use of a toroidal reflecting surface enhances the collection efficiency into a small target, such as an optical fiber, relative to a spherical reflecting surface by substantially reducing aberrations caused by the off-axis geometry. A second concave reflector is placed opposite to the first reflector to enhance further the total flux collected by a small target.

Additionally, more than one reflector may be used with a lamp. For example, dual reflectors or three or more reflectors, as taught in U.S. Pat. Nos. 5,706,376 and 5,862,277, may be incorporated into the preferred embodiment.

Notably, any number of lamps including low pressure, medium pressure, high pressure, and ultra high-pressure lamps, which are made of various materials, e.g., most commonly mercury (Hg) can be used with the system configuration according to the present invention, depending upon the blood or influent characteristics and flow rates through the system. Furthermore, while high and ultra high pressure lamps have not been used commercially to date by any prior art system, predominantly because of the low energy efficiency associated with them and the lack of capacity for prior art design and configuration formulas to include high pressure UV lamps, the present invention is advantageously suited to accommodate medium to high to ultra high pressure lamps, all of which can be metal, halogen, and a combination metal halide. Additionally, spectral calibration lamps, electrodeless lamps, and the like can be used.

In particular, by way of example and not of limitation, one preferred embodiment according to the present invention employs a light pump housing a pencil-type spectral calibration lamp. With a light pump, the number of lamps necessary to treat a given number of the blood purifiers can be reduced. Also, the lamps are not susceptible to fouling, since they are not exposed to the blood to be purified. Furthermore, the maintenance and servicing of the purifier is greatly simplified. The pencil-type spectral calibration lamps are compact and offer narrow, intense emissions, an average intensity that is constant and reproducible, and a longer life relative to other high wattage lamps. Hg (Ar) lamps of this type are generally insensitive to temperature and require only a two-minute warm-up for the mercury vapor to dominate the discharge, then 30 minutes for complete stabilization. A Hg(Ar) UV lamp, which is presently commercially available and supplied by ORIEL Instruments, is used in the preferred embodiment according to the present invention. The ORIEL Hg(Ar) lamp, model 6035, emits UV radiation at 254 nm. When operated at 15 mA using a DC power supply, this lamp emits 74 microwatt/cm2 of 254 nm radiation at 25 cm from the source.

Another preferred embodiment according to the present invention employs medium to high-pressure UV lamps, more preferably high-pressure UV lamps. These lamps may include mercury and/or mercury halide lamps, such as Hg(Ar), Hg(Xe), and Hg(Ne).

The light generated by these sources is focused via optics and fibers that are joined by UV-transmissive optical couplers. By way of example and not of limitation, these couplers can be quartz, liquid-filled, hollow, or dielectric coaxial couplers.

The present invention advantageously includes all of the above features, in particular because the UV lamps are separated from the blood purifier and include a light delivery system that incorporates optical components. Without the use of optical components in combination with the UV light source, the intensity of the light could not be effectively focused, directed, and controlled to provide an efficacious disinfection because the UV dosage entering the blood purifier would not be great enough to sterilize the microorganisms. By using optical components incorporated into the blood purifier itself, the blood purifier need be coupled to only one fiber optic transmission line for the supply of UV light. Alternately, the fiber optic transmission line and blood purifier may be simply juxtaposed to allow irradiated of the blood purifier by the light exiting the transmission line or other optics.

The light pump arrangement beneficially extends the lamp life thereby providing a longer replacement time or lamp life cycle. Since turning the lamp off and on degrades the lamp life, the system can be constructed and configured such that other appliances and areas are sterilized intermittently with the blood purifier by simply routing the UV light to the device or area to be irradiated. Thus, the lamp need not be turned on and off frequently. However, a timer or other means of system activation can be incorporated into the blood purifier to control exposure.

The UV light source may be presented in at least two primary configurations: a vertical riser configuration and a planar or horizontal configuration. In the vertical riser configuration the UV light source is positioned above the fluid to be treated and projecting a UV dose zone downward toward and into the fluid to be treated, with the fluid moving upward toward the UV light source. Alternatively, the UV light source may be presented in a planar or horizontal design, wherein the UV light source is positioned above the fluid to be treated and projecting a UV dose zone downward toward and into the fluid to be treated, with the fluid moving in a direction substantially perpendicular to the UV dose zone.

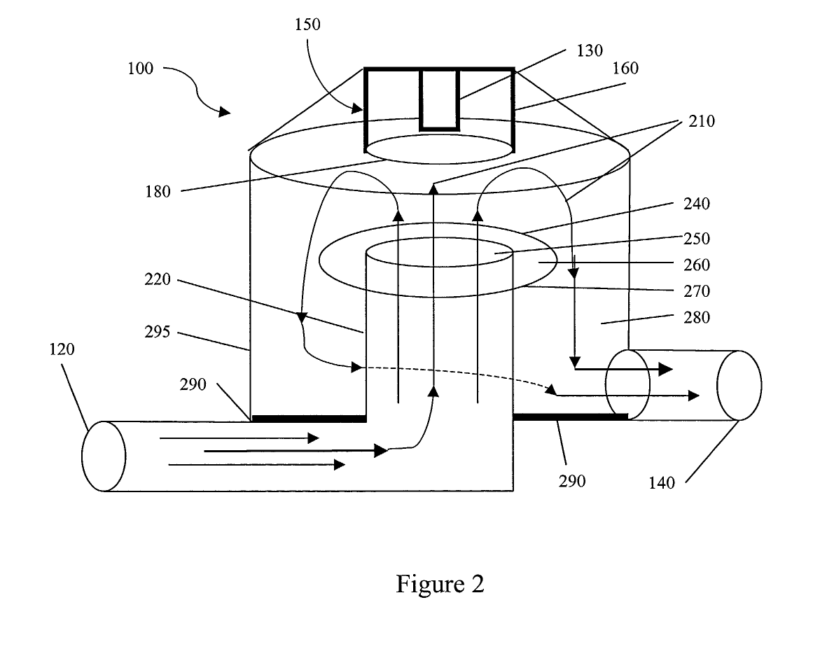

The UV light source may be presented in a vertical riser configuration according to a preferred embodiment of the present invention, as shown generally at 100 in FIG. 2, wherein the fluid enters into the vertical riser configuration (VRC) via a pipe or outlet 120 and passes therethrough prior to discharge from the pipe or outlet 140 for consumption or end use. Furthermore, the VRC includes at least one UV light source 130. This UV light source 130 is part of a lamp assembly, as shown generally at 150 in FIG. 2. The lamp assembly 150 is composed of a housing 160 that encases the UV light source 130, at least one optical component 180, and UV light ray output (not shown) that exits the housing. The UV light ray output exits the housing above the fluid 210 to be treated, this fluid entering the VRC through the inlet pipe 120 and being forced upward through the interior pipe 220 of the VRC 100 toward the UV light ray output that is projected downward toward the fluid surface and into the fluid 210 to be treated, once again with the fluid moving upward toward the UV light source 130. At least one interface plate 240 may be fitted to the top of the interior pipe 220, thus increasing the exposure time of the fluid 210 to the UV light ray output. The at least one interface plate 240 contains a hole or holes 250 that allows fluid rising upward through the interior pipe 220 to exit at the top of the pipe. The fluid then traverses across the superior surface 260 of the interface plate 240 to the plate edge 270, where it then descends into the exterior chamber 280 of the VRC. The fluid is prevented from returning into the interior pipe 220 by a base plate 290 that solidly connects the exterior of the interior pipe 220 with the interior of the outer pipe 295. The fluid then exits the VRC 100 through the pipe or outlet 140. The UV light rays may be projected downward from a UV light source or a lamp system that includes optical components. These optical components may include, but are not limited to, reflectors, shutters, lenses, splitters, focalizers, mirrors, rigid and flexible light guides, homogenizer or mixing rods, manifolds and other couplers, filters, gratings, diffracters, color wheels, and the like. These optical components are internal to the lamp system and are positioned between the UV light source or lamp and the UV ray light output of the lamp assembly, thereby focusing, directing, and controlling the light ray output that irradiates the fluid and that sterilizes any microorganisms that exist in the fluid. The UV light ray output irradiates and may also be transmitted through the fluid. UV light ray output that is transmitted through the fluid and strikes the reflective interior surfaces (not shown) of the VRC components is reflected back into the fluid where it may strike microorganism. The reflection of the UV light ray output back into the fluid by the reflective interior surfaces of the VRC components enhances the killing capacity of the VRC system.

Several UV dose zones are established within the VRC system. The first zone is the air UV dose zone which occurs just beneath the UV light source and just above the blood and the at least one interface plate. The next zone is the interface plate UV dose zone which occurs at the intersection of the water and the at least one interface plate. The at least one interface plate is used to provide a surface zone for UV disinfection above the fluid and to provide additional treatment means for balancing pH, affecting effluent chemistry, providing a catalyst, and the like. The last zone is the submerged UV dose zone, which creates a variable UV dose zone that decreases in effectiveness at greater distances from the UV light source.

Alternatively to the vertical configuration, the UV light source may be presented in a planar or horizontal design, as shown generally at 300 in FIG. 1, wherein at least one UV transmission line 18 that connects to the blood purifier 20 at a portal 22, which may alternatively be at least one portal if more than one light input is desired. The blood purifier portal is equipped with optical components, or portal optics, 32 that further control the UV light at the blood purifier 20 in order to provide additional focus and/or control of the UV light for the disinfection of the blood 24. The portal optics project the UV light, creating a UV dose zone, onto the blood which is flowing past in a perpendicular manner from the influent point 37 in a direction substantially perpendicular to the UV light source toward the effluent point 38. The dose zone and the housing may be equipped with UV reflective optical components, or interior optics 26, and may also be composed of a UV reflective interior surface and/or coating 28. For longevity as well as UV reflectivity, the interior surfaces may be made of a UV reflective material selected from the group consisting of UV reflective metals, e.g., stainless steel, aluminum, or the like. In the preferred embodiment, the blood purifier is made to be disposable for single-use applications. Additionally, the contribution of the reflectance of internal surfaces to the efficacy of the system can be capitalized upon by incorporating UV reflective materials and reflection enhancing two- and three-dimensional design into the blood purifier. Moreover, additional surfaces to enhance reflectance may be added to the purifier zone. More particularly, the blood purifier and other components form an integrated 2- and 3-dimensional design that incorporates UV-reflectant materials, design, and surfaces that advantageously enhance the efficacy of the system.

Several UV dose zones are established within the system. The first zone is the air UV dose zone, which occurs just beneath the UV light source and just above the blood. The next zone is the air/blood interface UV dose zone, which occurs at the air and blood interface. The last zone is the submerged UV dose zone, which occurs within the flowing blood.

A key factor in the design of a UV disinfection system and method according to the present invention involves the integration of two main components, including the non-submerged UV light source system and the hydraulic system. The hydraulic system includes a hydraulic tube and pumping system for forcing the fluid through the tube toward the light source(s). The present invention includes the use of hydraulic systems that comprise a transporter or pumping system, and at least one interface plate. The hydraulic system serves at least three functions: it carries blood to the UV dose region, regulates the flow to the UV dose region, and discharges the treated blood to a container.

Such an embodiment is easily scalable. For example, the size of the embodiment may extend from a small, portable application with a single point of UV irradiation to a large, multipoint application.

In the preferred embodiment, at least one portal optic is positioned at the portal opening of the blood purifier, between the portal opening and the blood purifier. The function of the at least one portal optic is to control the distribution of UV light in the blood purifier in order to enhance the UV disinfecting and degrading capacity of the system. The portal optics may be similar to those described for the source optics, including but not limited to reflectors, shutters, lenses, splitters, mirrors, rigid and flexible light guides, homogenizer or mixing rods, manifolds and other couplers, filters, color wheels, and the like, can be utilized in combination to achieve the desired control and output, as set forth in U.S. Pat. Nos. 6,027,237; 5,917,986; 5,911,020; 5,892,867; 5,862,277; 5,857,041; 5,832,151; 5,790,725; 5,790,723; 5,751,870; 5,708,737; 5,706,376; 5,682,448; 5,661,828; 5,559,911; D417,920 and co-pending applications Ser. Nos. 09/523,609; 09/587,678; 09/630,245; 09/723,679; 09/723,731; 09/724,068; 09/724,180; and 09/723,733, which are commonly owned by the assignee of the present invention. Additionally, optical component such as gratings, dichroic filters, focalizers, gradient lenses, and off-axis reflectors may be used. Finally, side-emitting fiber optic transmission lines may be used to distribute the UV light over the filter.

All UV transmissive optical components for the portal optics are made of UV-transmissive material and all UV-reflective optical components for the portal optics are made of UV-reflective material. These optics may extend into the blood purifier. For example, fiber optic transmission lines may be incorporated into the blood purifier and used to route UV light to the various areas of the blood purifier. The fiber optic lines may include quartz fibers, side-emitting fibers, glass fibers, acrylic fibers, liquid core fibers, hollow-core fibers, core sheath fibers, dielectric coaxial fibers, or a combination of fibers. The optics may also be incorporated into the structure of the blood purifier. For example, the interior of the blood purifier may be of a UV reflective material such that UV radiation striking these surfaces is reflected back through the blood.

Such a system of UV disinfection can be easily integrated into the blood purification function cycle by activating the UV light source or allowing irradiation of the blood purifier interior at a predetermined time in the blood purification function cycle. Alternately, the UV disinfection system may be manually activated when desired or may be programmed to activate when blood is detected.

Such a device has several advantages. First, the disinfected blood is completely free from microorganisms without requiring the addition of chemicals or other additives that would increase the chemical residue in the blood. Next, the use of removeably connectable portal optics to separate the light source from the fluid container allows for continuous use of the light source without the need for disinfection of the light source after the disinfection of every container of fluid. This extends the lamp life significantly. Also, the system can be used to disinfect blood as it is being collected, as the dose delivery device can be inserted in the blood collection line prior to the collection container and UV light routed to the dose delivery device with fiber optic transmission lines. By disinfecting blood at collection, the loss of blood due to bacterial contamination at collection can be prevented. In fact, because the primary contamination of blood is from the core of skin pushed into the needle at insertion, the intensity of light during the first few seconds of blood collection can be greatly increased to sterilize the core of skin before it has a chance to contaminate all the blood. Moreover, use of a light pump in such an application will allow for the collection of blood from multiple persons or animals simultaneously. Such an arrangement would eliminate the need for a lamp or light source at every point of application. Because it may not be necessary to continuously irradiate each point of application, such an arrangement would allow the same size lamp as would be require for a single application to service multiple applications intermittently and/or on demand, thus utilizing the lamp more efficiently. Additionally, placing the lamp exterior to the application reduces the risk of glass and/or mercury contaminating the blood should the lamp or lamp housing break.

Certain modifications and improvements will occur to those skilled in the art upon a reading of the foregoing description. By way of example, various optical components are used depending upon the particular UV light source or lamp selection for a given system. Moreover, a wide range of applications are contemplated within the scope of the present invention, including application of the UV blood purification system and method to purifiers involved in the manufacture of biological products and the like.

All modifications and improvements have been deleted herein for the sake of conciseness and readability but are properly within the scope of the following claims.

I claim:

1. A blood purification system for the effective sterilization of microorganisms, the system comprising at least one light source connected by at least one optical connection positioned to provide a focused, controllable light output to a blood purifier, and a control mechanism, thereby producing at least one UV dose zone for the effective sterilization of microorganisms in a blood.

2. The blood purification system according to claim 1, wherein the light source is a light pump including at least one lamp, at least one optic, a housing, and a power supply.

3. The blood purification system according to claim 1, wherein the light source is at least one lamp.

4. The blood purification system according to claim 3, wherein the lamp is a UV lamp.

5. The blood purification system according to claim 4, wherein the UV lamp is a high-intensity lamp.

6. The blood purification system according to claim 4, wherein the UV lamp is a spectral calibration lamp.

7. The blood purification system according to claim 4, wherein the UV lamp is an electrodeless lamp.

8. The blood purification system according to claim 4, wherein the UV lamp is a mercury halide lamp.

9. The blood purification system according to claim 4, wherein the UV lamp emits light in the UVV and UVC wavelengths.

10. The blood purification system according to claim 4, wherein the light source includes at least one light source optical component positioned to provide a focused, controllable light output to a blood purifier.

11. The blood purification system according to claim 10, wherein the light source optical component is UV transmissive.

12. The blood purification system according to claim 10, wherein the light source optical component is UV reflective.

13. The blood purification system according to claim 10, wherein the at least one light source optical component is selected from the group consisting of reflectors, shutters, lenses, splitters, focalizers, mirrors, rigid and flexible light guides, homogenizer, mixing rods, manifolds and other couplers, filters, gratings, diffracters, gradient lenses, color wheels, off-axis reflectors, cascading reflectors, splitting reflectors, and combinations thereof.

14. The blood purification system according to claim 1, wherein the at least one optical connection is a fiber optic transmission line.

15. The blood purification system according to claim 14, wherein the fiber optic transmission line is removably connectable to the light source and the blood purifier.

16. The blood purification system according to claim 1, wherein the fiber optic transmission line is selected from the group of fiber optic transmission lines including acrylic lines, glass lines, liquid core lines, quartz lines, hollow core lines, core-sheath lines, dielectric coaxial lines, and combination thereof.

17. The blood purification system according to claim 1, wherein the blood purifier includes a dose zone and a housing.

18. The blood purification system according to claim 17, wherein the housing is UV reflective.

19. The blood purification system according to claim 17, wherein the dose zone includes a portal for removable connection to a fiber optic transmission line.

20. The blood purification system according to claim 19, further including at least one portal optical component positioned between the portal opening and the interior of the blood purifier.

21. The blood purification system according to claim 20, wherein the at least one portal optical component is UV transmissive.

22. The blood purification system according to claim 20, wherein the at least one portal optical component is UV reflective.

23. The blood purification system according to claim 20, wherein the at least one portal optical component is selected from the group consisting of reflectors, shutters, lenses, splitters, focalizers, mirrors, rigid and flexible light guides, homogenizer, mixing rods, manifolds and other couplers, filters, gratings, diffracters, gradient lenses, color wheels, off-axis reflectors, cascading reflectors, splitting reflectors, and combinations thereof.

24. The blood purification system according to claim 1, wherein the blood purifier uses enhanced two-dimensional design to improve the blood purification.

25. The blood purification system according to claim 1, wherein the blood purifier uses enhanced three-dimensional design to improve the blood purification.

26. The blood purification system according to claim 17, wherein the dose zone includes a delivery device.

27. The blood purification system according to claim 26, wherein the delivery device includes at least one light emitter selected from the group consisting of side-emitting fiber optic transmission lines, end-emitting fiber optic transmission line, and combinations thereof.

28. The blood purification system according to claim 26, wherein the delivery device is a vertical riser configuration (VRC) in which the blood is moved at a predetermined rate toward the UV light output, thereby producing an increasing UV dose within the blood as it approaches the light output.

29. The blood purification system according to claim 28, wherein the vertical riser configuration system is scalable to applications.

30. The blood purification system according to claim 26, wherein the delivery device is a planar configuration in which the blood is moving at a predetermined rate perpendicular to the UV light output, thereby producing a constant UV dose within the blood as it moves through the delivery device.

31. The blood purification system according to claim 28, wherein the blood purifier is manufactured from a material selected from the group consisting of acrylic, plastic, quartz, glass, and combinations thereof.

32. The blood purification system according to claim 28, wherein the blood purifier is disposable.

33. The blood purification system according to claim 1, wherein at least one interior surface of the blood purifier is a UV reflective surface.

34. The blood purification system according to claim 33, wherein the at least one UV reflective surface is selected from the group consisting of aluminum, stainless steel, and combinations thereof.

35. The blood purification system according to claim 1, wherein the interior of the blood purifier includes at least one interior optical component that is attached to the interior surfaces.

36. The blood purification system according to claim 35, wherein the at least one interior optical component is UV transmissive.

37. The blood purification system according to claim 35, wherein the at least one interior optical component is UV reflective.

38. The blood purification system according to claim 35, wherein the at least one interior optical component is selected from the group consisting of reflectors, shutters, lenses, splitters, focalizers, mirrors, rigid and flexible light guides, homogenizer, mixing rods, manifolds and other couplers, filters, gratings, diffracters, gradient lenses, color wheels, off-axis reflectors, cascading reflectors, splitting reflectors, and combinations thereof.

39. A blood purifier for the effective sterilization of microorganisms in a blood, the blood purifier including a dose zone and housing, thereby producing at least one dose region for the effective sterilization of microorganisms in a blood.

40. The blood purifier system according to claim 39, wherein the housing is UV reflective.

41. The blood purifier according to claim 39, wherein the housing includes a portal for removable connection to a fiber optic transmission line.

42. The blood purifier according to claim 39, further including at least one portal optical component positioned between the portal and the interior of the blood purifier.

43. The blood purifier according to claim 42, wherein the at least one portal optical component is UV transmissive.

44. The blood purifier according to claim 42, wherein the at least one portal optical component is UV reflective.

45. The blood purifier according to claim 42, wherein the at least one portal optical component is selected from the group consisting of reflectors, shutters, lenses, splitters, focalizers, mirrors, rigid and flexible light guides, homogenizer, mixing rods, manifolds and other couplers, filters, gratings, diffracters, gradient lenses, color wheels, off-axis reflectors, cascading reflectors, splitting reflectors, and combinations thereof.

46. The blood purifier according to claim 39, wherein the blood purifier uses enhanced two-dimensional design to improve the blood purification.

47. The blood purifier according to claim 39, wherein the blood purifier uses enhanced three-dimensional design to improve the blood purification.

48. The blood purifier according to claim 39, wherein the dose zone further includes a delivery device.

49. The blood purifier according to claim 48, wherein the delivery device includes at least one light emitter selected from the group consisting of side-emitting fiber optic transmission lines, end-emitting fiber optic transmission line, and combinations thereof.

50. The blood purification system according to claim 48, wherein the delivery device is a vertical riser configuration (VRC) in which the blood is moved at a predetermined rate toward the UV light output, thereby producing an increasing UV dose within the blood as it approaches the light output.

51. The blood purification system according to claim 50, wherein the vertical riser configuration system is scalable to applications.

52. The blood purification system according to claim 48, wherein the delivery device is a planar configuration in which the blood is moving at a predetermined rate perpendicular to the UV light output, thereby producing a constant UV dose within the blood as it moves through the delivery device.

53. The blood purification system according to claim 50, wherein the blood purifier is manufactured from a material selected from the group consisting of acrylic, plastic, quartz, glass, and combinations thereof.

54. The blood purification system according to claim 50, wherein the blood purifier is disposable.

55. The blood purification system according to claim 39, wherein at least one interior surface of the blood purifier is a UV reflective surface.

56. The blood purification system according to claim 55, wherein the at least one UV reflective surface is selected from the group consisting of aluminum, stainless steel, and combinations thereof.

57. The blood purification system according to claim 39, wherein the interior of the blood purifier includes at least one interior optical component that is attached to the interior surfaces.

58. The blood purification system according to claim 58, wherein the at least one interior optical component is UV transmissive.

59. The blood purification system according to claim 58, wherein the at least one interior optical component is UV reflective.

60. The blood purification system according to claim 58, wherein the at least one interior optical component is selected from the group consisting of reflectors, shutters, lenses, splitters, focalizers, mirrors, rigid and flexible light guides, homogenizer, mixing rods, manifolds and other couplers, filters, gratings, diffracters, gradient lenses, color wheels, off-axis reflectors, cascading reflectors, splitting reflectors, and combinations thereof.

61. A method for the effective sterilization of microorganisms in blood, comprising the steps of: providing at least one UV light source connected by at least one optical connection positioned to provide a focused, controllable light output to a blood purifier, and a control mechanism, thereby producing at least one UV dose zone for the effective sterilization of microorganisms in a blood; activating the UV light source, passing the blood through the blood purifier, thereby providing sterilized blood.
